(19) United States
(12) Patent Application Publication
(10) Pub. No.: US 2022/0339230 A1
(43) Pub. Date: Oct. 27, 2022

(54) COMPOSITION COMPRISING BILBERRY EXTRACT AS ACTIVE INGREDIENT FOR PREVENTING OR TREATING SENSORINEURAL HEARING IMPAIRMENT

(71) Applicant: IMDPHARM INC., Suwon-si, Gyeonggi-do (KR)

(72) Inventors: Young-Joon PARK, Seoul (KR); Sook CHOI, Seoul (KR)

(73) Assignee: IMDPHARM INC., Suwon-si, Gyeonggi-do (KR)

(21) Appl. No.: 17/639,908

(22) PCT Filed: Sep. 4, 2020
(86) PCT No.: PCT/KR2020/011961

§ 371 Date: Apr. 25, 2022

(30) Foreign Application Priority Data
Sep. 6, 2019 (KR) 10-2019-0110886

(51) Int. Cl. A61K 36/45; A61P 27/16

(52) U.S. Cl. CPC A61K 36/45; A61P 27/16; A61K 2236/331; A61K 2236/333

(57) ABSTRACT

The present invention relates to a composition comprising a bilberry extract as an active ingredient for preventing or treating sensorineural hearing impairment. The bilberry extract was found to exhibit effects of inhibiting the apoptosis of auditory cells in which sensorineural damage has been induced by neomycin and increasing a reduced number of hair cells in juvenile zebra fishes such that the composition comprising the bilberry extract as an active ingredient can be provided as a pharmaceutical composition and a health food for preventing or treating various hearing impairments induced by sensorineural damage.

### TECHNICAL FIELD

The present disclosure relates to a composition including a bilberry extract as an active ingredient for preventing or treating sensorineural hearing impairment.

### BACKGROUND ART

Hearing loss (hearing impairment) is the most common disease that 15-20% of the population may have. The number of people with hearing impairment is increasing due to environmental pollution and aging in modern society, and since auditory disorder is permanent, prevention is important before occurring.

Most of the hearing impairment is developed by environmental and genetic factors such as sudden, drug-related (antibiotics, anticancer drugs), noise-induced, traumatic, senile and congenital factors and caused by sensorineural hearing impairment that mainly brings damage and death of auditory cells. For the treatment of sensorineural hearing impairment, many signaling pathways involved in inner ear hair cell regeneration, proliferation of hair cells and differentiation thereof have been found, and with the development of technologies such as gene editing or cell transplantation, great progress has been made in the study of hair cell regeneration in recent years, but there is still little development of a clear mechanism for suppression and prevention of hearing impairment or development of preventive and therapeutic drugs.

In recent years, as society has been industrialized, the number of people with hearing impairment due to noise is also rapidly increasing. Noise-induced hearing impairment due to cultural and leisure activities as well as occupational noise-induced hearing impairment such as workers and military personnel working in noisy environments are increasing. According to the Industrial Accident Act of Korea, exposure to environmental noise of 90 dBA or higher may damage hearing. The US Occupational Safety and Health Administration (OSHA) implements noise control for noise environments with a noise level of 85 dBA or higher. It has also been reported that the human auditory organ is affected by noises above 75 dBA. Since the noise of 75 dBA is the noise level of roadside where cars pass by, it seems that everyone in the industrial society lives in the noise that is harmful to the auditory organ.

In addition to environmental noise, adolescents are frequently exposed to loud noises in their leisure life, including MP3 use, so noise-induced hearing impairment has recently appeared in all ages. Noise-induced hearing impairment in youth eventually damages hearing enough to be able to communicate only by using hearing aids from the age of 40, and the degree of hearing impairment becomes more severe when aging is proceeded. Auxiliary tools such as hearing aids also become less effective as the degree of hearing impairment increases, so severe hearing damage eventually causes serious communication problems. In other words, when the younger generation experiencing noise-induced hearing impairment becomes older, the degree of hearing impairment becomes more severe, and hearing impairment has enormous influence in determining the quality of life in various generations, from the elderly to the young.

Recently, as a study to find substances effective for the prevention and treatment of hearing impairment, preclinical studies on antioxidants, N-methyl-D-aspartate (NMDA) antagonists, apoptosis inhibitors and growth factors have been reported, but it has been shown that there are limitations to proceed with clinical tests. To date, there are no approved drugs for the prevention and treatment of noise-induced hearing impairment, and as reported in the academic world, only drugs in the preclinical stages and clinical stages such as Mg, N-acetylcysteine (NAC) and Ebselen have been reported.

On the other hand, a bilberry (Vaccinium myrtillus) is a perennial plant commonly found in all regions of Finland and a typical coniferous plant that continues to grow for up to 30 years through young branches, and the best time of gathering is from the end of July to the beginning of September. Vitamins C and E are contained in the bilberry, and as a source of medicinal fibers, wild bilberry has a dark blue flesh compared to similar highbush blueberry which is a cultivated plant.

### DISCLOSURE

### Technical Problem

The present disclosure provides a composition including a bilberry extract as an active ingredient to protect auditory cells and hair cells from sudden factors, noise, cellular aging, or ototoxic drugs such as anticancer drugs and antibiotics so as to provide a pharmaceutical composition and a health food for preventing or treating sensorineural hearing impairment.

### Technical Solution

The present disclosure provides a pharmaceutical composition for preventing or treating sensorineural hearing impairment including a bilberry extract as an active ingredient.

In addition, the present disclosure provides a health functional food for preventing or ameliorating sensorineural hearing impairment including a bilberry extract as an active ingredient.

### Advantageous Effects

According to example embodiments of the present disclosure, it has been found that a bilberry extract exhibits effects of inhibiting the apoptosis of auditory cells in which sensorineural damage has been induced by neomycin and increasing a reduced number of hair cells in zebrafish larvae and thus a composition including the bilberry extract as an active ingredient may be provided as a pharmaceutical composition and a health food for preventing or treating various types of hearing impairment induced by sensorineural damage.

### BRIEF DESCRIPTIONS OF THE DRAWINGS

FIG. 1 shows a result of MTT analysis observing an apoptosis inhibitory effect in auditory cell lines treated with 3 and 15 μg/mL of a bilberry extract.

FIG. 2 shows a result of determining whether the number of hair cells increases after treatment of 0.1 μg/mL of a bilberry extract in neomycin-pretreated zebrafish larvae.

FIG. 3 shows results of checking a hearing threshold using 8 kHz TB stimulation sound.

FIG. 4 shows results of checking a hearing threshold using 16 kHz TB stimulation sound.

FIG. 5 shows results of checking a hearing threshold using 24 kHz TB stimulation sound.

### BEST MODE

Hereinafter, example embodiments of the present disclosure will be described in more detail.

While conducting development research on preventive and therapeutic drugs for hearing impairment using natural materials with excellent stability in order to solve side effect issues of the conventional therapeutic drugs for hearing impairment, the inventors of the present disclosure completed the disclosure by confirming that a bilberry extract exhibited effects of inhibiting the apoptosis of auditory cells in which sensorineural damage has been induced by neomycin and increasing a reduced number of hair cells in zebrafish larvae.

The present disclosure may provide a pharmaceutical composition for preventing or treating sensorineural hearing impairment including a bilberry extract as an active ingredient.

The bilberry extract may be extracted with water, or C1 to C2 alcohol or an aqueous solution thereof, and more preferably extracted with ethanol or methanol solvents, but is not limited thereto.

The sensorineural hearing impairment may be selected from the group consisting of sudden hearing impairment, ototoxic hearing impairment, noise-induced hearing impairment, traumatic hearing impairment, senile hearing impairment, and congenital hearing congenital, but is not limited thereto.

The bilberry extract may inhibit the apoptosis of auditory cells and increase the number of hair cells.

The pharmaceutical composition may include 0.1 to 90 parts by weight of the bilberry extract based on 100 parts by weight of the total pharmaceutical composition.

In an example embodiment of the present disclosure, the pharmaceutical composition for preventing or treating hearing impairment including the bilberry extract as an active ingredient may use at least one formulation selected from the group consisting of injection agents, granules, discutients, tablets, pills, capsules, suppositories, gels, suspensions, emulsions, drops or liquids according to a conventional method.

In another example embodiment of the present disclosure, the pharmaceutical composition for preventing or treating hearing impairment including the bilberry extract as an active ingredient may further include one or more additives selected from the group consisting of carriers, excipients, disintegrants, sweeteners, coating agents, swelling agents, glydents, flavoring agents, antioxidants, buffers, bacteriostats, diluents, dispersants, surfactants, binders and lubricants which are appropriate to be commonly used in the preparation of pharmaceutical compositions.

Specifically, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil may be used as carriers, excipients and diluents, and solid preparations for oral administration include tablets, pills, powder, granules and capsules, wherein such solid preparation may be prepared by mixing at least one excipient in the composition, for example, starch, calcium carbonate, sucrose or lactose and gelatin. In addition to simple excipients, lubricants such as magnesium stearate and talc may also be used. Liquid preparations for oral use include suspensions, solutions, emulsions and syrups, and various excipients such as wetting agents, sweeteners, fragrances and preservatives may be included in addition to commonly used simple diluents such as water and liquid paraffin. Formulations for parenteral administration include aqueous sterile solutions, non-aqueous solutions, suspensions, emulsions, freeze-dried preparations and suppositories. As the non-aqueous solvents and suspensions, vegetable oil such as propylene glycol, polyethylene glycol and olive oil as well as injectable ester such as ethyl oleate may be used. As a base material for the suppositories, witepsol, macrogol, tween 61, cacao butter, laurinum and glycerogelatin may be used.

According to an example embodiment of the present disclosure, the pharmaceutical composition may be administered into a subject in a conventional manner via intravenous, intraarterial, intraperitoneal, intramuscular, intrasternal, transdermal, intranasal, inhalation, topical, rectal, oral, intraocular or intradermal routes.

The preferred dosage of the bilberry extract may vary depending on the condition and weight of a subject, the type and severity of the disease, a drug form and the route and duration of administration, and may be appropriately selected by a person skilled in the art. According to an example embodiment of the present disclosure, although not limited thereto, the daily dose may be 0.01 to 300 mg/kg, specifically 0.1 to 300 mg/kg, and more specifically 0.1 to 200 mg/kg. Administration may be conducted once a day or in several divided doses, but the scope of the present disclosure is not limited thereby.

In the present disclosure, the ‘subject’ may be a mammal including a human, but is not limited to these examples.

In addition, the present disclosure may provide a health food for preventing or ameliorating sensorineural hearing impairment including a bilberry extract as an active ingredient.

The health food may be used together with other foods or food additives other than the bilberry extract, and appropriately used according to a conventional method. The mixed amount of the active ingredient may be suitably determined depending on the purpose of use thereof, for example prophylactic, health or therapeutic treatment.

The effective dose of compounds contained in the health food may be used according to the effective dose of the therapeutic drug, but in the case of long-term intake for the purpose of health and hygiene or health control, effective dose may be less than or equal to the above range while it is clear that the compounds may be used in an amount beyond the above range because there is no problem in terms of safety.

The type of health food is not particularly limited, and examples may include meat, sausage, bread, chocolate, candy, snacks, confectionery, pizza, ramen, other noodles, gum, dairy products including ice cream, various soups, beverages, tea, drinks, alcoholic beverages, and vitamin complexes.

The “sensorineural hearing impairment” of the present disclosure occurs when inner ear components or accompanying nerve components are affected and the components may include nerves or sensory components when the auditory nerve or auditory nerve pathway of the brain is affected. Sensorineural hearing impairment may be hereditary, or it may be due to acoustic trauma (e.g., very loud noises such as explosions), viral infection, drug-induction or Meniere's disease. Neural hearing impairment may result from brain tumors, infections, or various brain and nerve disorders such as stroke. Some genetic disorders, such as Refsum's disease (defective accumulation of branched fatty acids) may also cause neurological disorders that affect hearing impairment. The auditory nerve pathway is damaged by demyelinating diseases such as idiopathic inflammatory demyelinating disease (including multiple sclerosis), transverse myelitis, Devic's disease, progressive multifocal leukoencephalopathy, Guillain Barre syndrome, chronic inflammatory demyelinating polyneuropathy and anti-MAG peripheral neuropathy.

### MODES FOR CARRYING OUT INVENTION

Hereinafter, examples will be described in detail to help the understanding of the present disclosure. However, the following examples are merely illustrative of the content of the present disclosure, and the scope of the present disclosure is not limited to the following examples. The examples of the present disclosure are provided to more completely explain the present disclosure to those skilled in the art.

### <EXAMPLE> PREPARATION OF BILBERRY EXTRACT

### Example 1: Preparation of Ethanol Extract I

100 g of bilberry was added to 0.5 to 1 L of a 90 (v/v) % aqueous ethanol solution and stirred at 0 to 40° C. at 1600 rpm for 2 hours for primary extraction. The extract from the primary extraction was transferred and stored, 0.5 to 1 L of a 70 (v/v) % aqueous ethanol solution was added to the remaining residue, then the mixture was stirred at 0 to 40° C. at 1600 rpm for 2 hours for secondary extraction, and the secondary extract was transferred and stored. Then, 0.5 to 1 L of the 70 (v/v) % aqueous ethanol solution was added to a second residue, and the mixture was tertiarily extracted in the same method.

After mixing the primary, secondary and tertiary extracts, alcohol was evaporated while the mixture was being concentrated at a temperature of 50° C. or less. The concentrate obtained by the above process was diluted to 1.5±5% Brix and then centrifuged at 6900 rpm for 6 to 12 minutes.

The supernatant obtained via centrifugation was purified by compression filtration. Thereafter, 1200 L of water was added to the purified solution for primary washing for 2 hours, 5000 L of 70 (v/v) % ethanol was added to the primarily washed purified solution for secondary washing, and the washed solution was collected and concentrated.

Dried products obtained by spray-drying the concentrate obtained by the above process were dissolved in water to an appropriate concentration and used in the experiment.

### Example 2: Preparation of Ethanol Extract II

After pulverizing frozen bilberry flesh, 100 g of the pulverized product was immersed in 0.5 to 1 L of the 70 (v/v) % aqueous ethanol solution and extracted at 28 to 30° C. Then, the supernatant was separated by centrifugation, citric acid was added to the separated supernatant, and the precipitate produced thereby was removed for purification. After the remaining extract was concentrated, ethanol was added for dilution, and the extract was dried at 57 to 60° C. for 30 to 36 hours.

The bilberry extract dried by the above process was dissolved in water to an appropriate concentration and used in the experiment.

### Example 3: Preparation of Methanol Extract

After pulverizing the frozen bilberry flesh, 100 g of the pulverized product was immersed in 0.5 to 1 L of a 70 (v/v) % aqueous methanol solution and extracted at 28 to 30° C. Then, the supernatant was separated by centrifugation, citric acid was added to the separated supernatant, and the precipitate produced thereby was removed for purification. After the remaining extract was concentrated, ethanol was added for dilution, and the extract was dried at 57 to 60° C. for 30 to 36 hours.

The bilberry extract dried by the above process was dissolved in water to an appropriate concentration and used in the experiment.

### <Experimental Example> Confirmation of Hearing Impairment Treatment Effect of Bilberry Extract

1. Confirmation of Apoptosis Inhibitory Effect of Auditory Cell Lines

In order to confirm the hearing impairment treatment effect of the bilberry ethanol extract prepared in Example 1, the effect of the bilberry extract on the apoptosis of auditory cell lines was checked.

First, an auditory cell line (House-Ear Institute-organ of Corti 1; HEI-OC1) expressing auditory genes was cultured in high concentration glutamine-added Dulbecco's modified Eagle's Medium (DMEM) in which 10% fetal bovine serum (FBS) and 50 U/mL of interferon gamma are contained in the presence of 10% CO₂ at 33° C.

Then, a sensorineural hearing impairment cell line was established by treating neomycin (NM) to induce sensorineural damage.

After the sensorineural hearing impairment cell line was pretreated with 3 and 15 μg/mL of the bilberry ethanol extract prepared in the same process as in Example 1 for 1 hour, 15 mM of neomycin (NM) was treated for 24 hours, and the apoptosis effect by neomycin was quantitatively checked via MTT (Duchefa Biochemistry, M 1415.0001, Netherlands) assay.

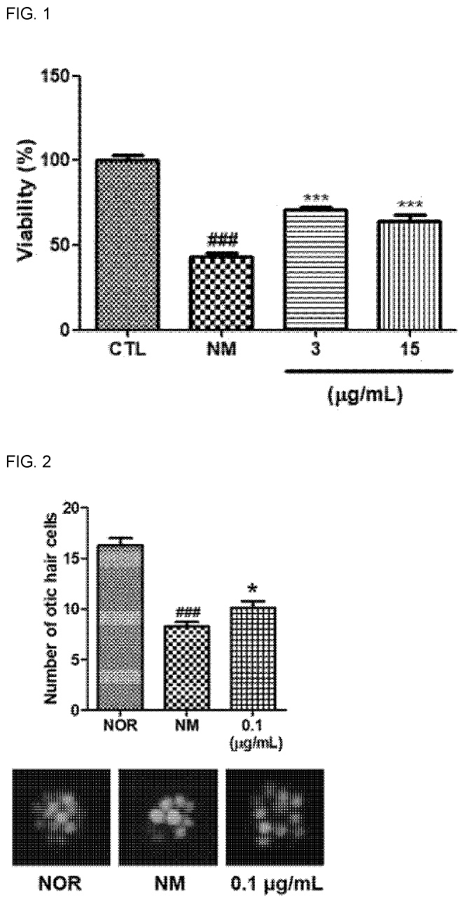

As a result, as shown in FIG. 1, it was confirmed that the cell group treated with 3 and 15 μg/mL of the bilberry ethanol extract had the effect of inhibiting auditory cell death due to neomycin.

2. Confirmation of Hair Cell Protective Effect in Zebrafish Model

In order to confirm the hearing impairment treatment effect of the bilberry extract prepared in Example 1, the effect of the bilberry extract on hair cells reduced by neomycin was checked.

First, zebrafish larvae obtained 6 days after fertilization were placed in 24 wells and exposed to 2 μM of neomycin for 1 hour.

Then, with the replacement with 0.1 μg/mL of the bilberry ethanol extract, the zebrafish larvae were exposed for 6 hours, wherein a 0.03% sea salt solution was treated as a control group.

For direct observation of hair cells, zebrafish larvae were anesthetized with 0.02% tricaine and stained with 0.1% YO-PRO for 30 minutes. Hair cells were checked with a fluorescence microscope (Olympus 1×70, Olympus, Japan), and the data were analyzed by counting the number of hair cells obtained by the fluorescence microscope.

As a result, it was confirmed that the number of hair cells of the zebrafish larvae treated with the bilberry ethanol extract was significantly increased compared to the control group as shown in FIG. 2.

### <Experimental Example 2> Confirmation of Hearing Impairment Ameliorating Effect after Exposure to Noise

1. Hearing Threshold Determination Using Click Sound as Broadband Stimulation Sound

To confirm the effect of the bilberry extract on a hearing threshold after exposure to noise, a hearing threshold measurement experiment using auditory brainstem response was conducted.

Auditory brainstem response (ABR) measurement is a method of evaluating a response to a sound by measuring electrical energy when a sound stimulus is transmitted as an electrical signal from auditory nerves. The response when sound reaches the auditory nerve by passing through an outer ear, middle ear, and cochlea reflects all the state of the outer ear, middle ear, and cochlea, thereby reflecting the actual sound energy reaching the brain. The hearing threshold refers to a minimum sensory point of barely audible sound, and in the case of normal mice, a response is observed even to a sound as low as 20 dB on average.

Specifically, five groups of 10-12 mice respectively were divided into mice to be administered with 50, 100 and 200 mg/kg of the bilberry extract and mice as an untreated control group and then evaluated. The noise was exposed for 1 hour with a 115 dB compound sound, 50, 100 and 200 mg/kg of the bilberry ethanol extract prepared as in Example 1 were orally administered 24 hours after the noise exposure, and oral administration was conducted at the same time every day. The hearing threshold was evaluated before exposure to noise and days 1, 7, and 14 after exposure.

For the auditory brainstem response test, the mice were anesthetized with ketamine (4.57 mg/kg) and xylazine (0.43 mg/kg) by intramuscular injection and then evaluated while the body temperature was maintained at 37±0.5° C. In the auditory brainstem response test, the stimulation sound was evaluated by gradually lowering by 5 dB from 80 dB using a click sound, which is a broadband stimulation sound, and the lowest sound showing a response was set as a threshold.

1-1. Hearing Threshold Determination Using 8 kHz TB Stimulation Sound

The auditory brainstem response test was performed in the same manner as above, and evaluation was conducted by gradually lowering the sound by 5 dB from 80 dB using an 8 kHz pure tone as a stimulation sound.

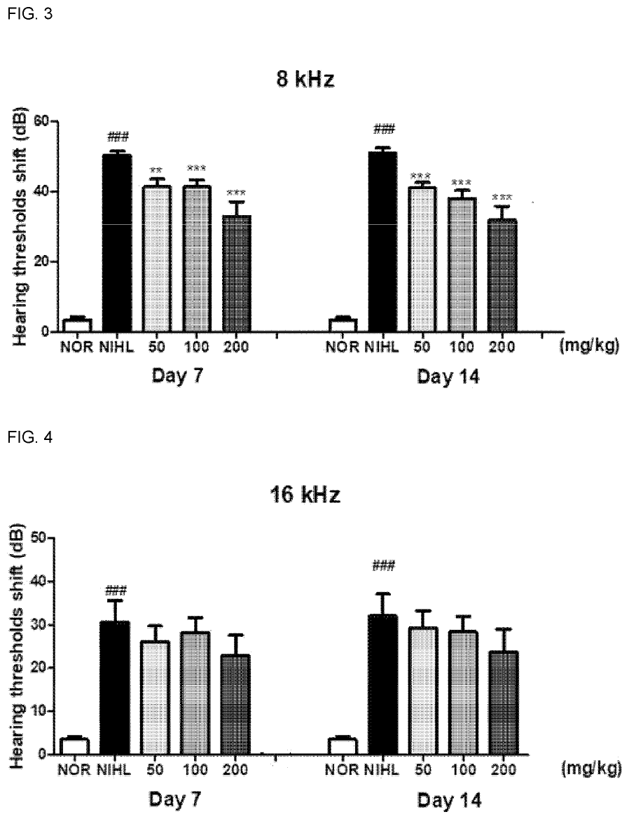

As a result, it was confirmed that hearing was improved after exposure to noise depending on the dose of the bilberry ethanol extract as shown in FIG. 3.

1-2. Hearing Threshold Confirmation Using 16 kHz TB Stimulation Sound

The auditory brainstem response test was performed in the same manner as above, and evaluation was conducted by gradually lowering the sound by 5 dB from 80 dB using a 16 kHz pure tone as a stimulation sound.

As a result, it was confirmed that hearing was improved after exposure to noise depending on the dose of the bilberry ethanol extract as shown in FIG. 4.

1-3. Hearing Threshold Confirmation Using 24 kHz TB Stimulation Sound

The auditory brainstem response test was performed in the same manner as above, and evaluation was conducted by gradually lowering the sound by 5 dB from 80 dB using a 24 kHz pure tone as a stimulation sound.

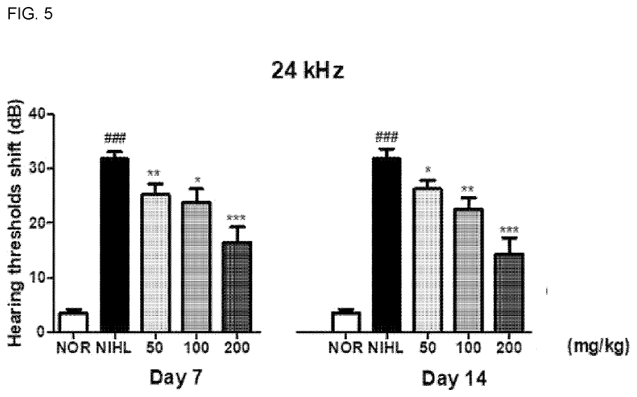

As a result, it was confirmed that the hearing was improved after exposure to noise depending on the dose of the bilberry ethanol extract as shown in FIG. 5.

Although specific parts of the present invention have been described in detail above, it is clear for those skilled in the art that these specific descriptions are merely preferred example embodiments and the scope of the invention is not limited thereto. Accordingly, the substantial scope of the invention will be defined by the appended claims and equivalents thereof.

1. A method of preventing or treating sensorineural hearing impairment, comprising:
administering a pharmaceutical composition comprising a bilberry extract as an active ingredient to a subject.

2. The method of claim 1, wherein the bilberry extract is extracted with water, or C1 to C2 alcohol or an aqueous solution thereof.

3. The method of claim 1, wherein the sensorineural hearing impairment is selected from the group consisting of sudden hearing impairment, ototoxic hearing impairment, noise-induced hearing impairment, traumatic hearing impairment, senile hearing impairment, and congenital hearing impairment.

4. The method of claim 1, wherein the bilberry extract inhibits apoptosis of auditory cells and increases the number of hair cells.

5. The method of claim 1, wherein the pharmaceutical composition comprises 0.1 to 90 parts by weight of the bilberry extract based on 100 parts by weight of the total pharmaceutical composition.

6. A health food for preventing or ameliorating sensorineural hearing impairment comprising a bilberry extract as an active ingredient.
